(19) United States
(12) Patent Application Publication
(10) Pub. No.: US 2009/0311138 A1
(43) Pub. Date: Dec. 17, 2009

(54) SANITIZATION OF AIRCRAFT OR VEHICLE CABIN

(76) Inventor: Peter Klaptchuk, Regina (CA)

Correspondence Address: STANDLEY LAW GROUP LLP, 6300 Riverside Drive, Dublin, OH, 43017, US

(21) Appl. No.: 12/162,918

(22) PCT Filed: Jan. 31, 2007
(86) PCT No.: PCT/CA2007/000128

§ 371 Date: Jan. 20, 2009

(30) Foreign Application Priority Data
Jan. 31, 2006 (CA) 2,534,700

(51) Int. Cl. A61L 2/20; A61L 9/00

(52) U.S. Cl. 422/30; 422/28

(57) ABSTRACT

A method of sanitizing a passenger cabin includes excluding people from the passenger cabin and substantially sealing the passenger cabin such that air inside the passenger cabin is prevented from exiting the passenger cabin. An ozone enriched atmosphere is created inside the passenger cabin by generating ozone and directing the ozone into the passenger cabin to increase an ozone concentration of the atmosphere inside the passenger cabin to at least a minimum concentration. The ozone enriched atmosphere is circulated through the passenger cabin and air circulation system thereof for a sanitation period calculated to be sufficient for sanitation. The ozone concentration of the atmosphere inside the passenger cabin is then reduced to a level safe for human occupation.

This invention is in the field of sanitization and in particular relates to a method for sanitizing passenger compartments such as aircraft cabins or other vehicle cabins such as buses.

### BACKGROUND

Recently there have been questions about whether passengers, pilots and cabin crews are at risk from exposure to contaminated air. Airlines want to improve the quality of re-circulated cabin air for the protection of passengers and crews from exposure to contaminants such as viruses, bacteria, and volatile organic compounds such as hydraulic fluids, engine lubricants, pesticides, jet fuels and de-icing compounds. With global travel increasing and with the potential for airborne transmission of viruses such as SARS or Asiatic flu that are capable of causing epidemics, it is obvious that the sanitization of the air and surfaces in airplane and vehicle cabins is vital to the health of passengers and crew. Other passenger carrying vehicles such as buses have similar concerns.

Such cabins typically contain fabrics, closely spaced seating, overhead compartments, washrooms, galleys and are difficult and time consuming to sanitize using conventional methods such as antiseptic cleaners and the like. Pathogens such as germs and viruses can remain in the cabin and infect passengers and crew. Air in passenger cabins is circulated through air circulation systems that include vanes in heating and air conditioning units which are difficult to access for cleaning and sanitation.

The use of ozone is well known as a disinfectant or sterilizing agent. Ozone is a powerful oxidizer which effectively kills microorganisms. Applications in water and waste treatments are well documented. In addition to the cost effectiveness and strong oxidizing power of ozone, the penetrating property of ozone makes it an ideal aerial disinfectant as well as a surface disinfectant of equipment, furniture, carpets, curtains, seat and wall coverings. Ozone is a useful disinfectant of rooms or enclosed spaces and their surfaces because as a gas it readily penetrates every corner and crevice of an enclosed space.

Ozone (O₃) is an unstable gas comprising three atoms of oxygen. It is unstable because ozone gas will readily degrade back to its stable state, diatomic oxygen (O₂), the form of oxygen humans breath to live, with the formation of free oxygen atoms or free radicals. The free oxygen atoms are highly reactive and will oxidize almost everything (including viruses, fungi, moulds, bacteria, organic and inorganic compounds). Ozone's high level of oxidation properties means that in addition to being a disinfectant, ozone is capable of eliminating odors caused by animals, smoke, perfume and fuel. Following sanitization with ozone, the sanitized space will be left with a clean, fresh smell. Ozone is considered an environmentally friendly disinfectant because it is a potent disinfectant at low concentrations, it does not produce any harmful residues and all residual ozone used in disinfection is converted back to oxygen within a relatively short period of time.

Ozone generators are presently available which are marketed to be used continuously in an enclosed space and in the presence of humans and animals to remove odors and freshen the air. However, the level of ozone generated by these ozone generators is low, since the ozone concentrations must be maintained at levels which will not adversely affect occupants.

It is generally accepted that the maximum ozone concentration in an atmosphere occupied by humans for any significant length of time is 0.100 parts per million (ppm), and preferably ozone concentration is below 0.050 ppm.

### SUMMARY OF THE INVENTION

It is an object of the present invention to provide a method of, and apparatus for, sanitizing aircraft and like vehicle passenger cabins that overcomes problems in the prior art.

The invention provides a method of sanitizing a passenger cabin, the passenger cabin including an air circulation system operative to circulate air through the passenger cabin. The method comprises excluding people from the passenger cabin and substantially sealing the passenger cabin such that air inside the passenger cabin is substantially prevented from exiting the passenger cabin; creating an ozone enriched atmosphere inside the passenger cabin by generating ozone and directing the ozone into the passenger cabin to increase an ozone concentration of the atmosphere inside the passenger cabin to at least a minimum concentration; circulating the ozone enriched atmosphere through the passenger cabin and air circulation system for at least a period of time equal to a sanitation period; and reducing the ozone concentration of the atmosphere inside the passenger cabin to a level safe for human occupation.

The ozone rich atmosphere inside the cabin is maintained at a high ozone concentration for a sufficient time period to in-activate pathogens. The atmosphere is circulated through the cabin in order to ensure the ozone rich atmosphere enters into corners and smaller spaces, and also circulates through the cabin's air circulation system in order to sanitize the ducts, heating and cooling vanes through which the air is typically circulated. The ozone level in the cabin is monitored remotely to insure the desired ozone concentration is maintained, and once the time period has been reached, the ozone is typically exhausted to the atmosphere where same will break down into oxygen. Fresh air can be drawn in and circulated through the cabin and air circulation system to push the ozone rich atmosphere out through the exhaust, and the ozone concentration monitored until same is safe for occupation. Alternatively, or in addition, the ozone rich atmosphere can be circulated through a catalyst to break the ozone down into oxygen and reduce the ozone concentration to safe levels.

An apparatus for practicing the method can comprise an ozone generator, a blower, and an ozone concentration sensor. Aircraft passenger cabins are typically sealed such that same may be pressurized to maintain air inside the cabin at satisfactory pressures when operating at high altitudes, and also include an air circulation system. In such aircraft it is convenient to seal the aircraft cabin and connect the blower to the air circulation system. The blower is connected at an inlet thereof to draw air from the cabin through the blower and the blower outlet is connected to direct the air back into the cabin. Ozone is generated and directed into the circulating air and the concentration of ozone in the air is measured. Since the cabin is sealed, air is maintained inside the cabin and the ozone concentration will rise to the desired level and be circulated through the cabin penetrating into corners and smaller spaces.

The ozone generator can be connected to the blower inlet or simply placed inside the cabin. The ozone concentration is monitored remotely and the ozone generator can be controlled to maintain the desired concentration of ozone, or in some situations it may be desired to simply operate the ozone generator at full capacity and raise the ozone level to the highest possible level. The ozone sensor is required to ensure that at least a minimum concentration is attained from which it will be possible to calculate the time required for sanitization.

When the required time has elapsed, the intake of the blower can be disconnected from the air circulation system and connected to draw in fresh air, while the end of the air circulation system that was connected to the blower outlet during sanitization can then be connected to an exhaust vent configured to discharge into the outside atmosphere at an elevated level away from any occupied areas where the ozone can safely break down. The blower is then operated to draw in fresh air and flush the ozone rich atmosphere out of the cabin and away through the exhaust vent. When the ozone concentration in the cabin is reduced to levels safe for human occupation, the generator, sensor, and blower are removed and normal aircraft operations can commence.

Alternatively and more conveniently in many instances, the blower, sensor, and ozone generator can be incorporated into a portable unit that is placed into the cabin. The vehicle air circulation system is operated to circulate the ozone rich atmosphere through heating and ventilation units to sanitize same, and the blower and circulation system cause the air inside the cabin to circulate into corners and small spaces, and the ozone concentration is timed and monitored as described above. When the desired time has elapsed, the air is circulated through a catalyst that causes the ozone to breakdown into oxygen, and when the ozone levels have been reduced to safe working levels, the unit is removed and the sanitation is complete.

The portable ozone sanitization unit is of use in the sanitization of airplane or vehicle cabins, and particularly the air circulation systems thereof which conventional cleaning processes typically do not reach. The timer and ozone concentration sensor on the unit provide safety to the maintenance personnel and to the subsequent passengers and staff who may be occupying the airplane or other vehicle cabin following sanitization.

### DESCRIPTION OF THE DRAWINGS

While the invention is claimed in the concluding portions hereof, preferred embodiments are provided in the accompanying detailed description which may be best understood in conjunction with the accompanying diagrams where like parts in each of the several diagrams are labeled with like numbers, and where:

FIG. 1 is a schematic side view of an aircraft cabin connected to an ozone sanitizing apparatus of the invention set up to add ozone to the atmosphere inside the aircraft cabin, and circulate the ozone rich atmosphere through the cabin;

FIG. 2 is a schematic side view of the aircraft cabin of FIG. 1 connected to an ozone sanitizing apparatus of the invention set up to flush the ozone rich atmosphere out of the cabin and out through an exhaust;

FIG. 3 is a schematic side view of an integrated ozone sanitizing apparatus with a catalytic ozone destruction unit, with a gate positioned so that air drawn in by the blower is not exposed to the catalyst;

FIG. 4 is a schematic side view of the intake of the integrated ozone sanitizing apparatus of FIG. 3 with the gate positioned so that air drawn in by the blower is exposed to the catalyst and broken down into oxygen gas.

### DETAILED DESCRIPTION OF THE ILLUSTRATED EMBODIMENTS

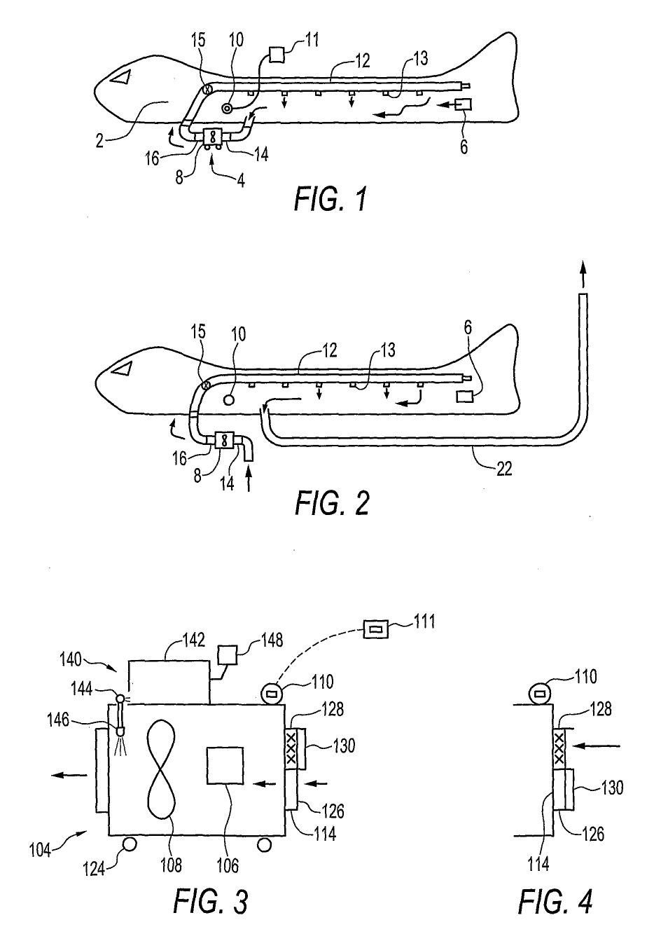

FIG. 1 schematically illustrates an aircraft passenger cabin 2 connected to an ozone sanitizing apparatus 4 of the invention set up to add ozone to the atmosphere inside the aircraft passenger cabin 2, and circulate the ozone rich atmosphere through the cabin 2. The sanitizing apparatus 4 comprises an ozone generator 6, a blower 8, and an ozone concentration sensor 10. Typically an oxygen concentrator will be incorporated into the ozone generator 6 to maximize the ozone generated.

People, and any desirable animals or the like, are excluded from the passenger cabin and the passenger cabin 2 is sealed in the same manner as such cabins are sealed for operation at high altitude so that air is prevented from exiting the passenger cabin 2. The blower 8 is connected to the air circulation system 12 of the aircraft. The circulation system 12 typically comprises a fan 15 to circulate air through ducts with outlets 13 spread out through the cabin 2. The blower 8 is connected at an inlet 14 thereof to draw air from the cabin 2 through the blower 8 and the blower outlet 16 is connected to direct the air back into the cabin 2 through the air circulation system 12. The ozone generator 6 generates ozone inside the cabin 2 to raise the concentration of ozone in the circulating air to a desired minimum level, and the concentration of ozone in the circulating air is measured at the sensor 10.

Since the cabin 2 is sealed, air is maintained inside the cabin and the ozone concentration will rise to the desired level and be circulated through the cabin penetrating into corners and smaller spaces. The blower 8 increases circulation of the air through the cabin 2 during the sanitation process, compared to the lesser circulation normally provided by the fan 15 of the cabin circulation system 12, and ensures that the ozone enriched air reaches all corners of the cabin.

The ozone generator 6 could also be connected to the blower inlet 14 if that is more convenient. The ozone concentration is monitored remotely and the ozone generator 6 can be controlled to maintain the desired concentration of ozone, or it may be desired to simply operate the ozone generator 6 at full capacity and raise the ozone level to the highest possible level. The ozone sensor 10 is required to ensure that at least a minimum concentration is attained from which it will be possible to calculate the time required for sanitization. Remote reading of the ozone concentration is provided by an ozone indicator 11 connected to the sensor 10 that can be located external to the cabin 2.

Generally it is contemplated that the time required for sanitization will be at most a few hours which will be coordinated to coincide with an overnight stop such as is common for commercial passenger aircraft.

In the embodiment of FIG. 1, when the required time has elapsed, and as illustrated in FIG. 2, the inlet 14 of the blower 8 is disconnected from the interior of the cabin 2 and connected to draw in fresh air, and the interior of the cabin 2 is connected to an exhaust vent 22 configured to discharge into the outside atmosphere at an elevated level, or at least in a location away from any occupied areas where the ozone can safely break down. The blower 8 is then operated to draw in fresh air and flush the ozone rich atmosphere out of the cabin 2 and away through the exhaust vent 22. When the ozone concentration in the cabin 2 is reduced to levels safe for human occupation, the generator 6, sensor 10, and blower 8 are removed and normal aircraft operations can commence.

With an ozone generator 6 of a given capacity, the ozone concentration attainable will be higher or lower depending on the size of the cabin 2 which correlates directly to the volume of air therein. Similarly the air moving capacity for effective circulation and flushing will vary with the size of the cabin 2. Typically the blower 8 and ozone generator 6 will have air moving and ozone generating capacities designed for aircraft of the different sizes in which they will be used.

Alternatively the ozone generator 106 could be integrated into a portable ozone sanitizing apparatus 104, as illustrated in FIG. 3, that is placed into the vehicle cabin. The illustrated apparatus 104 comprises an ozone sensor 110 and blower 108 to increase circulation of the air through the cabin during the sanitation process, compared to the lesser circulation normally provided by the conventional cabin circulation system, and ensure that the ozone enriched air reaches all corners of the cabin. The apparatus 104 could be mounted on wheels 124 or other wise conveniently movable. The air circulation system of the aircraft is operated during the sanitization process to circulate the ozone rich atmosphere through the heating and air conditioning units to destroy pathogens collected on the vanes and like parts of such units. The ozone concentration is monitored at a remote ozone indicator 111 connected to the ozone sensor 110, and once the desired level has been attained for the desired time period the ozone is degraded back to oxygen by circulating the air through a catalyst such as manganese dioxide in a thermal-catalytic destruct unit.

The illustrated apparatus 104 includes an inlet 114 that is divided into an open portion 126 and a catalyst portion 128. A gate 130 is movable from the position illustrated in FIG. 3 where incoming air drawn in by the blower 108 flows through the open portion 126 and the catalyst portion 128 is blocked, to the position illustrated in FIG. 4 where the open portion 126 is blocked and incoming air flows through the catalyst portion 128. Thus during the sanitation period, the ozone generator is operated to maintain the ozone concentration at the desired level, and the gate is in the position of FIG. 3 such that incoming air is not exposed to the catalyst. When the sanitation period is finished, the ozone generator is stopped, and the gate is moved to the position of FIG. 4 such that incoming air passes through the catalyst in the catalyst portion, and the ozone in the air is broken down into oxygen. When the ozone sensor 10 indicates that the ozone concentration has been reduced to a safe level, the apparatus 104 can be shut off and removed. While the ozone could be left to break down over time, the catalyst speeds up this natural break down and allows the process to be completed faster.

For example, manganese dioxide in a thermal-catalytic destruct unit catalytically converts the ozone back to oxygen gas. The process is exothermic and often produces enough beat to make the reaction go very quickly.

Reaction of the free oxygen atoms from the ozone to in-activate pathogens is somewhat increased where the atmosphere is relatively moist compared to a drier atmosphere. Thus increasing the relative humidity of the ozone rich atmosphere can increase the effectiveness of the method of the present invention.

For example, the portable ozone sanitizing apparatus 104 can be provided with a humidifier system 140 with a tank 142 containing water, and a pump 144 operative to pump water through a fogging nozzle 146 to create a mist or fog in the air-stream created by the blower 108 to increase the humidity of the atmosphere in the passenger cabin. A humidity sensor 148 can be provided to sense the relative humidity and operate the humidifier system 140 if required.

In a typical sanitization system for practicing the method of the invention, ozone and humidity monitoring, and subsequent adjustments would be controlled by a computer.

In smaller aircraft, rail cars, buses, or like vehicles, the cabin would be sealed to the extent possible, and parked outside where escaping ozone would not harm any persons, since leakage would be greater from ground vehicles and lower flying small aircraft that are not normally required to be well sealed. The vehicle air circulation system would be operated and the blower on the sanitation apparatus would cause the air inside the cabin to circulate into corners and small spaces, and the ozone concentration would be timed and monitored as described above.

It is contemplated that raising ozone concentrations to 4 to 5 ppm over a sanitation period of about one hour will allow time to ensure that the ozone rich air has circulated to all parts of the cabin and satisfactorily sanitize a passenger cabin. Higher concentrations and time periods may be used where there is some extra concern with respect to sanitation. Generally, where a lower concentration of ozone is present, the sanitation period should be longer, and where the concentration is higher, the sanitation period can be lower. Monitoring the ozone concentration during the period allows the sanitation period to be extended if for some reason the concentration falls below the minimum concentration desired for the sanitation period being used.

The sanitizing apparatus 104 can be remotely activated and monitored, either through wired or wireless communication so that the maintenance people can commence the sanitization process from outside the plane or vehicle cabin. The apparatus may have a local or remote visual or sound indication of operation so that personnel will be warned that the unit is in use and that it is not safe to enter the area that is being sanitized.

The foregoing is considered as illustrative only of the principles of the invention. Further, since numerous changes and modifications will readily occur to those skilled in the art, it is not desired to limit the invention to the exact construction and operation shown and described, and accordingly, all such suitable changes or modifications in structure or operation which may be resorted to are intended to fall within the scope of the claimed invention.

1. A method of sanitizing a passenger cabin, the passenger cabin including an air circulation system operative to circulate air through the passenger cabin, the method comprising:
excluding people from the passenger cabin and substantially sealing the passenger cabin such that air inside the passenger cabin is substantially prevented from exiting the passenger cabin;
creating an ozone enriched atmosphere inside the passenger cabin by generating ozone and directing the ozone into the passenger cabin to increase an ozone concentration of the atmosphere inside the passenger cabin to at least a minimum ozone concentration;
circulating the ozone enriched atmosphere through the passenger cabin and air circulation system for at least a period of time equal to a sanitation period; and
reducing the ozone concentration of the atmosphere inside the passenger cabin to a level safe for human occupation.

2. The method of claim 1 comprising circulating the ozone enriched atmosphere through the air circulation system by operating a fan of the air circulation system.

3. The method of claim 2 further comprising providing a blower and increasing the circulation of the ozone enriched atmosphere by operating the blower.

4. The method of claim 3 comprising connecting an intake of the blower to draw air from inside the passenger cabin and connecting an output of the blower to the air circulation system.

5. The method of claim 4 wherein the ozone concentration of the atmosphere inside the passenger cabin is reduced by connecting the intake of the blower to draw air from outside the passenger cabin and connecting the output of the blower to the air circulation system, and connecting an exhaust vent to the inside of the passenger cabin.

6. The method of claim 3 wherein the ozone concentration of the atmosphere inside the passenger cabin is reduced by providing a catalyst compartment on the blower and a catalyst contained in the catalyst compartment, wherein the catalyst is operative to catalytically convert ozone passing therethrough back to oxygen gas, and by configuring the blower to draw air from the passenger cabin through the catalyst.

7. The method of claim 1 wherein the ozone concentration of the atmosphere inside the passenger cabin is reduced by exhausting air inside the passenger cabin to the atmosphere and drawing fresh outside air into the passenger cabin to replace the exhausted air.

8. The method of claim 1 wherein the minimum ozone concentration is four parts per million, and the sanitation period is one hour.

9. The method of claim 8 wherein the minimum ozone concentration is greater than four parts per million, and the sanitation period is less than one hour.

10. The method of claim 8 wherein the minimum ozone concentration is less than four parts per million, and the sanitation period is greater than one hour.

11. The method of claim 1 further comprising increasing the relative humidity of the ozone rich atmosphere during the sanitation period.

12. The method of claim 1 where the passenger cabin comprises a portion of one of an aircraft, a bus, and a rail car.
